(19) United States
(12) Patent Application Publication
(10) Pub. No.: US 2003/0211583 A1
(43) Pub. Date: Nov. 13, 2003

(54) Egs molecules that specifically downregulae bcl-xl experession

(76) Inventor: Cy Stein, New City, NY (US)

Correspondence Address: John P White, Cooper & Dunham, 1186 Avenue of the Americas, New York, NY, 10036, US

(21) Appl. No.: 10/169,324

(22) PCT Filed: Jan. 3, 2001
(86) PCT No.: PCT/US01/00155

(51) Int. Cl. C12Q 1/68; C07H 21/04; A61K 48/00; C12P 19/34; C12N 15/88

(52) U.S. Cl. 435/91.2; 435/6; 514/44; 435/458; 536/23.2

(57) ABSTRACT

This invention provides a nuclease-resistant external guide sequence (EGS) oligonucleotide selected from the group consisting of Inno-1405, Inno-1407 and Bcl-xL1 whose sequences are set forth in Table 1. This invention also provides a method of inducing specific intracellular mRNA cleavage through activation of RNase P comprising contacting a cell with a complex comprising an EGS oligonucleotide and a carrier. This invention provides methods of downregulating PKC-α protein expression and PKC-α mRNA expression comprising contacting a cell with a complex comprising an EGS oligonucleotide and a carrier. This invention provides methods of downregulating bcl-xL protein expression and bcl-xL mRNA expression comprising contacting a cell with a complex comprising an EGS oligonucleotide and a carrier.

This application claims priority of U.S. Provisional Application No. 60/174,748, filed Jan. 3, 2000, the contents of which are hereby incorporated by reference.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

Most antisense oligonucleotide experiments are performed with molecules containing Rnase H-competent backbones. However, Rnase H may cleave nontargeted mRNAs bound to only partially complementary oligonucleotides. Decreasing such “irrelevant cleavage” would be of critical importance to the ability of the antisense biotechnology to provide accurate assessment of gene function. RNase P is a ubiquitous endogenous cellular ribozyme whose function is to cleave the 5′ terminus of precursor tRNAs to generate the mature tRNA. To recruit Rnase P, complementary oligonucleotides called external guide sequences (EGS), which mimic structural features of precursor tRNA, were incorporated into an antisense 2′-O-methyl oligoribonucleotide targeted to the 3′ region of the PKC-α mRNA. In T24 human bladder carcinoma cells, these EGSs, but not control sequences, were highly effective in downregulating PKC-α protein and mRNA expression. Furthermore, the downregulation is dependent on the presence of, and base sequence in, the T-loop. Similar observations were made with an EGS targeted to the bcl-xL mRNA.

Antisense technology is a commonly used experimental method to downregulate gene expression¹⁻³ and is also being used to develop therapeutics⁴. The antisense effect is thought to be mediated by RNase H^{5,6}, which cleaves the mRNA strand of an mRNA-DNA duplex⁷. RNase H activity is elicited by polyanionic oligodeoxyribonucleotides, such as nuclease-resistant phosphorothioates⁸. RNase H does not require a perfect duplex to cleave an mRNA, leading to the problem of “irrelevant cleavage” at nontargeted sites⁹⁻¹¹. A mere 4- to 7- base region of complementarity can lead to cleavage¹², and there are a large number of nested quartamer through heptamer sequence motifs in any 20-mer oligonucleotide. The extent of irrelevant cleavage is probably also a function of the quantity of oligonucleotide delivered to the nucleus¹³. Thus, in practice it may be difficult or impossible to determine precisely which genes are being cleaved by an antisense oligomer¹³.

Over the past decade, Altman and colleagues^{14,15} have developed the idea of eliciting mRNA cleavage by RNase P, the enzyme that cleaves the 5′ terminus of precursor tRNAs to generate the mature tRNA. A 32-mer synthetic complementary oligonucleotide (EGS) has been shown in cell-free systems to lead to RNase P-mediated cleavage of a target RNA¹⁶. The EGS has two hybridizing arms, the A-stem and D-stem, joined by a T-stem and T-loop (FIGS. 1A, B).

This construct mimics structural elements of a precursor tRNA, and the EGS-mRNA duplex can elicit RNase P activity, leading to target cleavage. Nuclease-resistant 2′-O-methyl oligoribonucleotides can be substituted at all positions, except at critical residues in the loop, without loss of activity¹⁶. In addition, the highly nuclease-sensitive ribopyrimidines found in the precursor tRNA loop can be replaced with less sensitive ribopurines¹⁷. The final oligomers are stable in 50% human serum for 24h¹⁶.

RNase P cleavage of chloramphenicol acetyltransferase (CAT) mRNA has been demonstrated in HeLa cells¹⁸, but the EGS was a 68-mer transcribed from a mouse U6 polIII promoter expressed off a transfected plasmid construct. This molecule is too long for scale-up synthesis, and because it is RNA, is easily hydrolyzable. Ma and colleagues¹⁶ have recently developed a series of nuclease-resistant, serum-stable EGSs that efficiently induce RNase P cleavage in vitro. However, it has never been demonstrated that exogenous administration of a chemically stabilized EGS can induce RNase P-mediated cleavage of a target mRNA in living cells. In this work, we use the established antisense PKC-α model in T24 bladder carcinoma cells^{19,20} to demonstrate that EGS can indeed perform this function in living cells, without the problems of irrelevant cleavage observed with the antisense oligonucleotide approach.

### SUMMARY OF THE INVENTION

This invention provides a nuclease-resistant external guide sequence (EGS) oligonucleotide selected from the group consisting of Inno-1405, Inno-1407 and Bcl-xL1 whose sequences are set forth in Table 1.

This invention provides a method of inducing specific intracellular mRNA cleavage through activation of RNase P comprising contacting a cell with a complex comprising an EGS oligonucleotide and a carrier.

This invention provides a method of downregulating PKC-α protein expression comprising contacting a cell with a complex comprising an EGS oligonucleotide and a carrier.

This invention provides a method of downregulating bcl-xL protein expression comprising contacting a cell with a complex comprising an EGS oligonucleotide and a carrier.

This invention provides a method of downregulating PKC-α mRNA expression comprising contacting a cell with a complex comprising an EGS oligonucleotide and a carrier.

This invention provides a method of downregulating bcl-xL mRNA expression comprising contacting a cell with a complex comprising an EGS oligonucleotide and a carrier.

### BRIEF DESCRIPTION OF THE FIGURES

FIGS. 1A-1C. (FIG. 1A) Structure of a precursor tRNA; the arrow indicates the natural RNase P cleavage point. (FIG. 1B) EGS bound to a target mRNA. Binding occurs through Watson-Crick interactions between the A-stem and D-Stem and the complementary sequence of the target. (From ref. 16, used with permission of the publisher. (FIG. 1C) Internalization of 5′-fluoreuroscein-labeled Inno-1405 (1 μM) complexed to Lipofectin (10 μg/ml) in T24 bladder carcinoma cells. Confocal microscopic images were obtained as described in the text. Shown is a maximum projection of all sections.

FIGS. 2A-2D. (FIG. 2A) Western blot analysis of PKC-α expression in T24 cells following treatment with EGS. Cells were treated with a complex of EGS (1 μM) and Lipotectin (10 μg/ml), and extracts (20 μg/lane) were prepared as described in Experimental Protocol. (FIG. 2B) Reproduction of the experiment in (A), with the addition of the control Inno-1414, in which the loop sequence 54-60 was reversed. As an additional positive control, in a, TMP (9 μM), and not Lipotectin, was used as the carrier for b, Isis 3521; 3 μM, an anti-sense PKC-α 20 mer all-phosphorothioate oligonucleotide. (FIG. 2C) Effects of treatment of T24 cells with various EGSs complexed with LipofectACE. Cells were treated with a complex of EGS (1 mM) and LipofectACE (10 μg/ml) for 4 h as described in the text. (FIG. 2D) Effects of various EGSs on the expression of PKC-α protein in 5637 bladder carcinoma cells.

FIGS. 3A-3B. Western blot analysis of proteins from T24 cells treated with various EGSs complexed to Lipofectin. (FIG. 3A) PKC-β1 expression. (FIG. 3B) PKC-ζ expression. The last lane is a positive control using Isos 3521 (a; 3 μM) complexed with TMP (b; 9 μM) which under these conditions will downregulate both PKC-α and PKC-ζ protein and mRNA expression, presumably by “Irrelevant cleavage”.

FIG. 4. Northern blot analysis of overexpression of the 8.5 and 4.2 kb PKC-α mRNAs following treatment of T24 cells with various EGSs complexed to Lipofectin. Membranes were probed with either a PKC-α or control GAPDH cDNA probe as described in the text.

FIGS. 5A-5B. (FIG. 5A) Western blot analysis of extracts (25 mg/lane) of T24 cells treated with the bcl-xL1 and bcl-xL2 EGS (1.5 μM) complexed to Lipofectin (10 μg/ml). (FIG. 5B) Northern analysis of T24 cells treated with bcl-xL1 and bcl-xL2 EGS complexed to Lipofectin (10 μg/ml) on the expression of the 2.7 kb bcl-x mRNA. Membranes were probed with either a bcl-xL or control GAPDH cDNA probe as described in the text.

### DETAILED DESCRIPTION OF THE INVENTION

This invention provides a nuclease-resistant external guide sequence (EGS) oligonucleotide selected from the group consisting of Inno-1405, Inno-1407 and Bcl-xL1 whose sequences are set forth in Table 1.

This invention provides a method of inducing specific intracellular mRNA cleavage through activation of RNase P comprising contacting a cell with a complex comprising an EGS oligonucleotide and a carrier.

In an embodiment of the above-described method of inducing specific intracellular mRNA cleavage through activation of RNase P the EGS oligonucleotide is of a 0.1 μM/ml to 1 μM/ml concentration and the carrier is of a 1 μg/ml to 30 μg/ml concentration. In another embodiment the carrier is a cationic lipid. In a further embodiment the cationic lipid is Lipofectin or LipofectACE. In yet another embodiment the EGS oligonucleotide is selected from the group consisting of Inno-1405, Inno-1407 and Bcl-xL1 whose sequences are set forth in Table 1. In a still further embodiment of the above-described methods the cell may be any human cell. In an embodiment the human cell may be selected from but not limited to the group consisting of a prostate cell, bladder cell, colon cell, breast cell, lung cell, endometrial cell, epithelial cell, ovarian cell, cervical cell, neural cell and blood cell. In another embodiment of the above-described methods the cell may be a cancer cell selected from but not limited to the group consisting of melanoma cells, basal cell carcinoma cells, squamous cell carcinoma cells, neuroblastoma cells, glioblastoma multiforme cells, myeloid leukemic cells, breast carcinoma cells, colon carcinoma cells, endometrial carcinoma cells, lung carcinoma cells, ovarian carcinoma cells, prostate carcinoma cells, bladder cancer cells, cervical carcinoma cells, osteosarcoma cells and lymphoma cells.

In another embodiment of the above-described method the RNase P does not cleave nontargeted mRNAs.

This invention provides a method of downregulating PKC-α protein expression comprising contacting a cell with a complex comprising an EGS oligonucleotide and a carrier.

In an embodiment of the above-described method the EGS oligonucleotide is selected from the group consisting of Inno-1405 and Inno-1407 whose sequences are set forth in Table 1. In another embodiment the EGS oligonucleotide is of a 0.1 μM/ml to 1 μM/ml concentration and the carrier is of a 1 μg/ml to 30 μg/ml concentration. In a further embodiment the carrier is a cationic lipid. In yet another embodiment the cationic lipid is Lipofectin or LipofectACE.

This invention provides a method of downregulating bcl-xL protein expression comprising contacting a cell with a complex comprising an EGS oligonucleotide and a carrier.

In an embodiment of the above-described method the EGS oligonucleotide is bcl-xL1 whose sequence is set forth in Table 1. In another embodiment the EGS oligonucleotide is of a 0.1 μM/ml to 1 μM/ml concentration and the carrier is of a 1 μg/ml to 30 μg/ml concentration. In a further embodiment the carrier is a cationic lipid. In yet another embodiment the cationic lipid is Lipofectin or LipofectACE.

This invention provides a method of downregulating PKC-α mRNA expression comprising contacting a cell with a complex comprising an EGS oligonucleotide and a carrier.

In an embodiment of the above-described method the EGS oligonucleotide is selected from the group consisting of Inno-1405 and Inno-1407 whose sequences are set forth in Table 1. In another embodiment the EGS oligonucleotide is of a 0.1 μM/ml to 1 μM/ml concentration and the carrier is of a 1 μg/ml to 30 μg/ml concentration. In a further embodiment the carrier is a cationic lipid. In yet another embodiment the cationic lipid is Lipofectin or LipofectACE.

This invention provides a method of downregulating bcl-xL mRNA expression comprising contacting a cell with a complex comprising an EGS oligonucleotide and a carrier.

In an embodiment of the above-described method the EGS oligonucleotide is bcl-xL1 whose sequence is set forth in Table 1. In another embodiment of the above-described method the EGS oligonucleotide is of a 0.1 μM/ml to 1 μM/ml concentration and the carrier is of a 1 μg/ml to 30 μg/ml concentration. In a further embodiment the carrier is a cationic lipid. In a still further embodiment of the above-described method the cationic lipid is Lipofectin or LipofectACE.

This invention will be better understood from the Experimental Details which follow. However, one skilled in the art will readily appreciate that the specific methods and results discussed are merely illustrative of the invention as described more fully in the claims which follow thereafter.

### RESULTS AND DISCUSSION

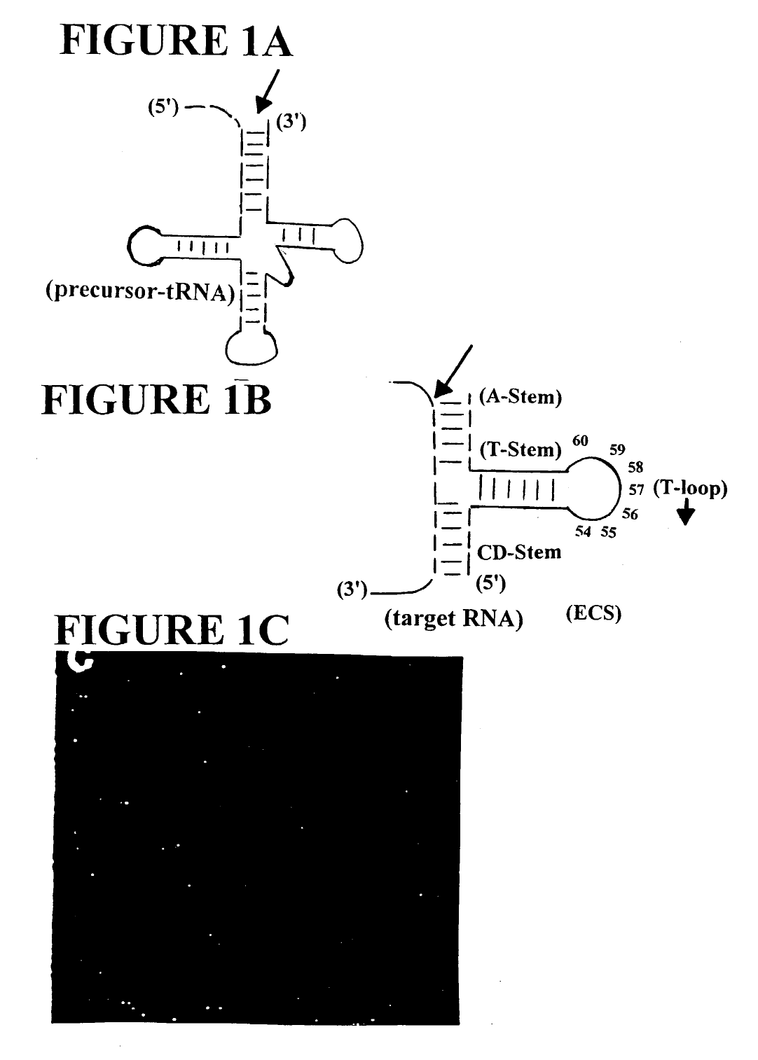

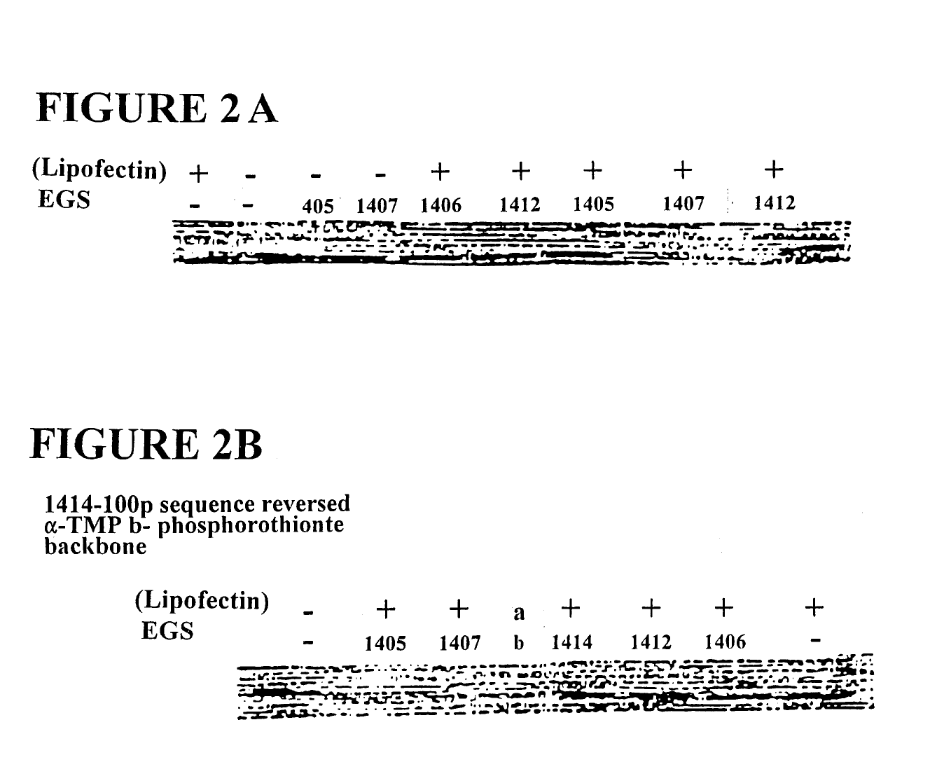

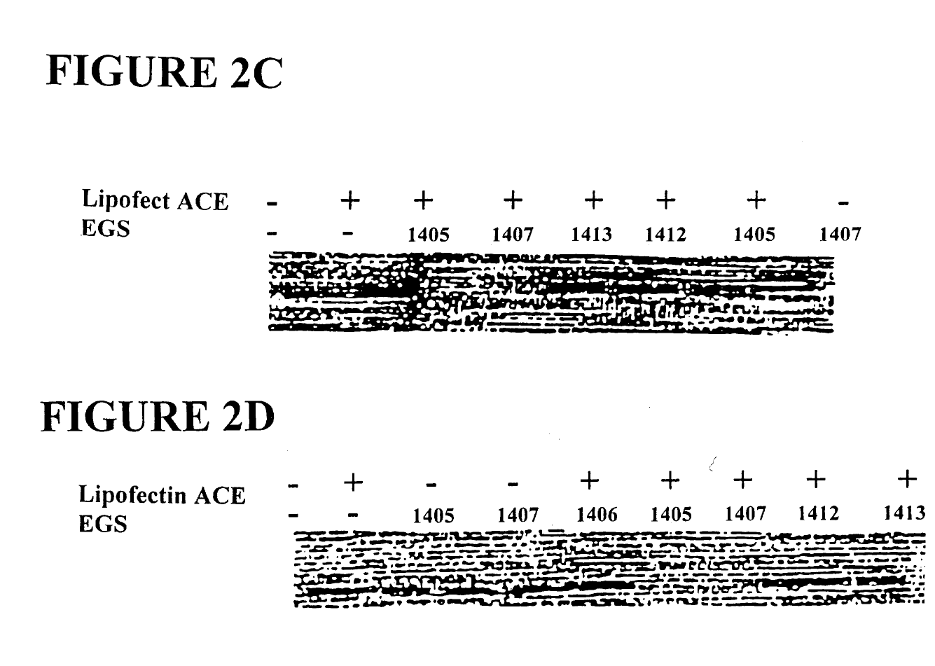

The EGS leads to specific cleavage of a target mRNA in mammalian cells. Isis 3521 is a 20-mer phosphorothioate oligonucleotide, targeted to the 3′ untranslated region (UTR) of the PKC-α mRNA, that has proved effective in downregulating protein and mRNA expression in human cells. Therefore, we designed oligomers to hybridize to the PKC-α mRNA at the Isis 3521 site (Table 1). T24 human bladder carcinoma cells were treated with the EGS constructs at various concentrations using Lipofectin or LipofectACE (both 10 μg/ml). Delivery of 1 μM Inno-1411 (5′-fluoresceinated Inno-1407) by Lipofectin is shown by confocal microscopy in FIG. 1C. Diffuse cytoplasmic staining and punctated intranuclear staining can be observed in virtually every cell. Western blot analysis of PKC-α expression was performed following treatment with EGS-Lipofectin (FIGS. 2A, B) or EGS-LipofectACE (FIG. 2C). Both Inno-1405 and Inno-1407 reproducibly decreased PKC-α protein expression (92%±8%; n=6). Inno-1405 lacks the 3′-terminal ACCA motif, demonstrating that the latter is not necessary for RNase P-mediated cleavage in mammalian cells. Inno-1406, which contains the 3′-terminal ACCA motif as unmodified RNA, is only slightly active, probably due to nuclease digestion (see FIG. 2B).

An EGS can be inactivated by deletion of specific nucleotides in the T-loop, complete substitution of the seven nucleotides in the T-loop with the 2′-OMe counterparts, disruption of the T-stem, or reversal of the T-loop sequence¹⁶. None of these alterations affected PKC-α protein expression (FIGS. 2,3). The controls included a species with 16 complementary 2′-O-methyl-modified ribonucleotides (Inno-1412), instead of the 14 present in Inno-1405 and 1407. Both Inno-1413, in which the necessary ribonucleotides in the loop of Inno-1405 were replaced by 2′-O-methyl ribonucleotides, and Inno-1414, with a reversed loop sequence relative to Inno-1405, were inactive (FIG. 2B). The latter is a critical control and strongly suggests that RNase P mediates the downregulation.

The concentrations of oligonucleotide and of Lipofectin (or LipofectACE) producing maximum downregulation were 1 μM and 10 μg/ml, respectively. Concentrations of either reagent that deviated by more than a factor or two from these values demonstrated greatly diminished activity. This is not surprising, as the dependence of antisense efficacy on dose (when the oligomer is delivered by cationic lipids) may be very narrow. This is probably due to properties of the lipid carrier and is presumably related to the nature of it interactions with endosomal membranes. Other transfection reagents complexed to these oligomers, for example the cationic porphyrin m-tetra(methylpyridyl) porphine (TMP) were ineffective, probably because the EGSs were too long for TMP-mediated transfection, which seems to be most effective when complexed with an oligomer of 20-mer length or less. As assessed by (3-[4,5-dimethylthiazol-2-yl]-2, 5-diphonyl tetrazolium bromide (MTT) assay, none of our oligonucleotides were cytotoxic.

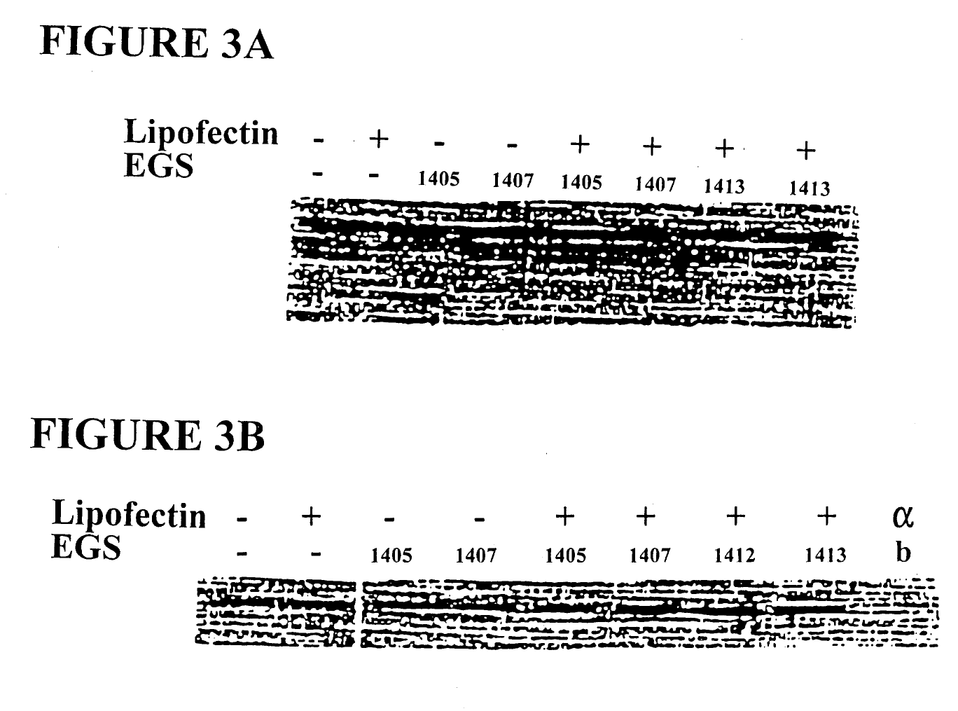

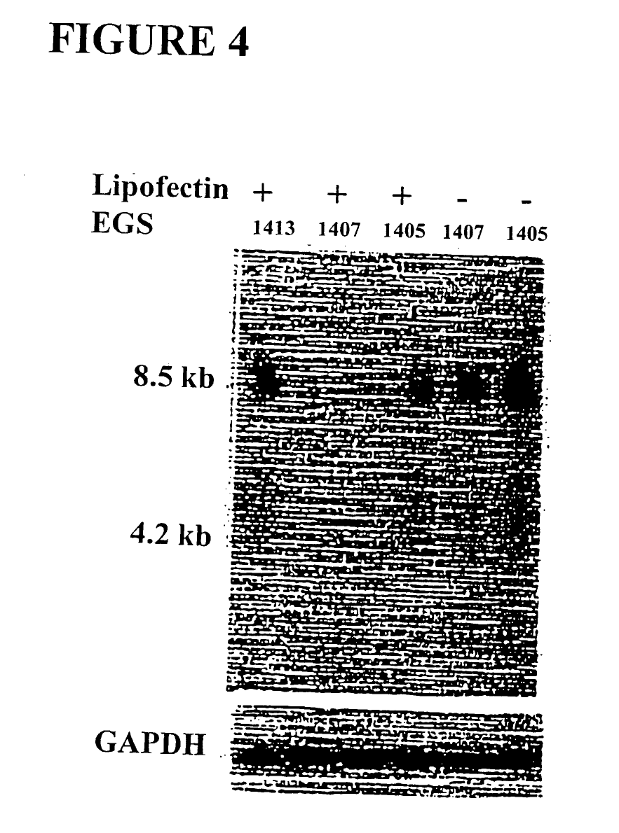

In previous studies, we used the phosphorothioate oligomer Isis 3521 delivered with TMP to downregulate PKC-α protein expression in T24 cells¹³. In our hands, the complex of Isis 3521 with Lipfectin was inefficient at downregulating PKC-α translation, although others have had better success. However, we also observed¹³ down regulation of PKC-ζ (see FIG. 3B), but not PKC-β1, -δ, or -ε. As there is an 11-base contiguous region of complementarity between Isis 3521 and the PKC-ζ mRNA, we assumed¹³ that the downregulation of PKC-ζ was due to irrelevant cleavage. However, in the current experiments Inno-1405 did not reduce PKC-ζ protein expression (FIG. 3B), although nine contiguous bases at the 5′ terminus are a perfect complement to the PKC-ζ mRNA. Presumably, PKC-ζ expression is unaffected owing to both the lack of RNaseII-mediated cleavage of its mRNA, and finer discrimination of duplex structure by RNase P. As an additional control we examined the expression of PKC-β1, which was also unaffected by treatment of cells with Inno-1405 (FIG. 3A). Further evidence of RNase-mediated activity comes from the results of northern analyses. Both Inno-1405 and -1407 dramatically reduce the expression of the 8.5 and 4.2 kB PKC-α mRNA transcripts (FIG. 4). A G3PDH control probe confirmed equivalent levels of RNA per lane and the absence of a general reduction of mRNA translation by the oligonucleotide-Lipofectin complex.

In our experience, T24 cells are an extremely reliable and reproducible test system to evaluate antisense technology. However, the activity of Inno-1405 and Inno-1407 also extends to 5637 human bladder carcinoma cells (FIG. 2D). This is consistent with the idea that RNase P recognizes a structural motif in Inno-1405 and Inno-1407, and cleaves the target PKC-α mRNA. As nucleotides in the T-loop seem to be recognized by RNase p^{21,22}, this idea is particularly credible in light of the observation that Inno-1414, which contained the identical hybridizing sequence, but with a reversed loop, is inactive. In addition, an RNase H mechanism of mRNA elimination can be ruled out because of the presence of 2′-O-methylated ribonucleotides, and steric blockade of translation seems to be unlikely because of the lack of activity of numerous control sequences, again including Inno-1414. We believe that this is the first demonstration in mammalian cells that an exogenous EGS can produce RNase P-mediated antisense effects. Furthermore, in sharp contrast to phophorothioate oligonucleotides; the EGS seem to downregulate PKC-α protein expression in the absence of RNase H-mediated irrelevant cleavage, although other factors, such as the intranuclear concentration of oligonucleotide, may also contribute to this absence.

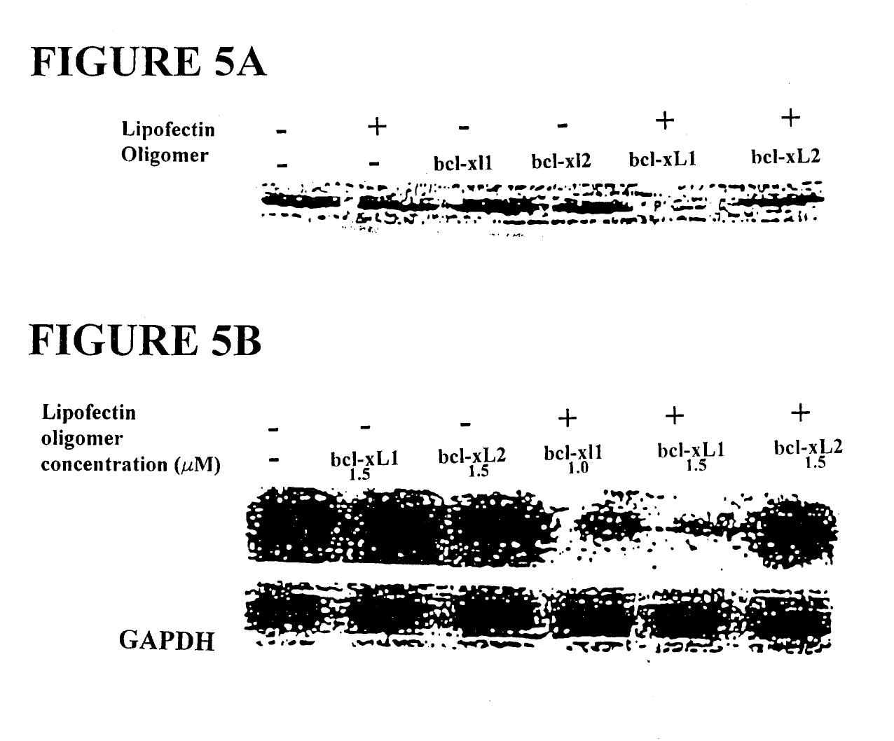

The EGS technology may be generally applicable. Bcl-xL is a strongly antiapoptotic protein that is expressed in T24 cells. When these cells were treated with a complex consisting of 1.5 μM bcl-xL1 and 10 μg/ml Lipofectin under identical conditions used for the downregulation of PKC-α, a dramatic, almost complete downregulation of bcl-xL protein expression was observed (FIG. 5). Northern analysis revealed a congruent diminution in bcl-x mRNA expression. Similar to what was observed in the case of PKC-α, reversal of the loop sequence (bcl-xL2) produced an inactive EGS oligomer. These observations, in combination with those on the EGS-induced downregulation of PKC-α expression, strongly suggest that RNase P is involved in the mechanism of action of EGS.

However, in order for the EGS methodology to become more widely used, ways must be found to develop shorter oligomers that can also elicit RNase P activity. Fortunately, there is preliminary evidence that this can in fact be accomplished²³⁻²⁴. In addition, the role of the carrier as a cofactor in oligonucleotide-mediated inhibition of protein translation needs to be thoroughly investigated. Nevertheless, EGS can assume a position in the growing collection of molecular tools that specifically modify gene expression in mammalian cells.

### EXPERIMENTAL PROTOCOL

Oligonucleotides. Oligonucleotides were prepared as described¹⁶. Briefly, 2′-O-Silyl-protected and 2′-O-methyl RNA phosphoramidites were purchased from PerSeptive Biosystems (Framingham, Mass.) with t-butylphenoxyacetyl as the exocyclic amine protective group. The EGS oligonucleotides were prepared on an Applied Biosystems (ABI, Foster City, Calif.) model 394 DNA/RNA synthesizer, 10 μM column). Standard synthesis reagents were purchased from commercial suppliers. The modified 3′-dimethoxytrityl-5′-succinate-dT) controlled pore glass (CPG) was prepared by ChemGene Corp. (Waltham, Mass.), and employed to provide a nuclease-resistant 3′-3′ linkage at the 3′ terminus of the molecule²⁵. Upon completion of fast deprotection in concentrated ammonium hydroxide/ethanol (NH₄OH/EtOH) (3:1 vol/vol) and desilylation in 1M tetrabutylammonium fluoride²⁶, crude 5′-DMT-containing EGS oligos were purified on reverse-phase HPLC. If the purity was less than 90%, as assessed by capillary gel electrophoresis (CGE; Beckman P/ACE system 5000), a second purification was conducted on anion-exchange HPLC. All materials that were used for subsequent studies in cell cultures were further characterized by CGE, analytical reverse-phase HPLC (Waters HPLC system using a Perkin-Elmer 3×3 C18 column, and matrix-assisted laser desorption-ionization-time-of-flight (MALDI-TOF) mass spectrometry (PE Biosystems, Voyager-DETM Biospectrometry Workstation). Isis 3521 was prepared as described¹⁵. The bcl-xL1 sequence was complementary to nucleotides 623-638 of the bcl-xL mRNA, and bcl-xL2 had the identical sequence but with the loop sequence reversed. This optimal sequence was chosen after screening forty, 18- and 20-mer randomly selected antisense phosphorothioate oligonucleotides with 100% complementarity to various regions of the bcl-xL mRNA. In the original EGS work by Altman et al.¹⁴, the four-nucleotide motif ACCA was added to the 3′ end of an EGS, mimicking all the natural tRNA precursors. However, recent studies have demonstrated that, at least in vitro, the 3′-ACCA could be deleted without compromising cleavage²⁵.

Cells. T24 and 5637 bladder carcinoma cells were obtained from American Type Culture Collection (Rockville, Md.), and were grown in McCoy's 5A medium (Life Technologies, Gaithersburg, Md.), containing 10% (vol/vol) heat-inactivated (56° C.) fetal bovine serum (FBS) (Life Technolgics), supplemented with 25 mM HEPES, 100 U/ml penicillin G sodium, and 100 μg/ml streptomycin sulfate. Stock cultures were maintained at 37° C. in a humidified, 5% CO₂ incubator.

Reagents. The anti-PKC-α monoclonal antibody (mAb) was purchased from Upstate Biotechnology, Lake Placid, N.Y. Anti-PKC-β1 or -ζ polyclonal antibodies were purchased from Life Technologies, and an anti-N-terminal bcL-x mAb was purchased from Santa Cruz (Santa Cruz, Calif.). Human PKC-α and bcl-xL cDNAs for northern analysis were generous gifts of Dr. I. B. Weinstein (Columbia University) and S. Korsmeyer (Washington University) TMP was obtained from Porphyrin Products (Logan, Utah).

Treatment of Cells with Oligonucleotide-Cationic Lipid Complexes. Cells were grown in six-well plates until −75% confluent. At this time, Lipofectin or LipfectACE (Life Technologies) was diluted in 100 μl of Opti-MEM medium (Life Technologies) with the EGS oligonucleotides to give a final concentration of 10 μg/ml lipid-l μMEGS, unless stated otherwise. The solutions were mixed gently and preincubated at room temperature for 30 min to allow the complexes to form. Then, 800 μl of opti-MEM media were added to the complexes, and the solution was mixed and overlaid onto the cells that had been rinsed with opti-MEM. The cells were then incubated at 37° C. for 7h, then washed and refed with complete McCoy's 5A media containing 10% PBS and allowed to incubate for an additional 19h before cell lysis and extract preparation. Complexes of Isis 3521 (3 μM) and TMP (9 μM) were prepared as described¹³.

Western Blotting. Cells treated with oligomer-cationic lipid or porphyrin complex were washed twice in cold PBS and then extracted in 100-150 μml of lysis buffer [50 mM Tris-HCl, pH 7.5; 1% NP-40; 0.25% sodium deoxycholate; 150 mM NaCl; 1 mM EGTA; 1 mM phenylmethylsulfonyl fluroide (PMSF); 1 mg/ml aprotinin, leupeptin; 1 mM Na₃VO₄; 1 mM NaF] at 4° C. for 30 min. Cell debris was removed by centrifugation at 14,000 g for 20 min at 4° C. Protein concentrations were determined using the Bio-Rad protein assay system (Bio-Rad Laboratories, Richmond, Calif.).

Aliquots of cell extracts containing 25-40 μg of protein were resolved by 10% sodium dodecyl sulfate-polyacrylamide gel electrophoresis (SHS-PAGE) and transferred to Hybond electrochemiluminescence (ECL) filter paper (Amersham, Arlington Heights, Ill.). Filters were incubated at room temperature for 1-2 h in Blotto 1 (5% nonfat milk powder in 50 mM Tris-HCl, pH 7.5, 200 mM NaCl, 0.1% Triton X-100) and then probed with a 1:500 dilution of a PKC isoform-specific (Upstate Biochemicals) or an N-terminal bcl-xL-specific (Santa Cruz) mAb in 1% BSA, 50 mM Tris-HCl, pH 7.5, 200 mM NaCl, and 0.02% NaN₅. Membranes were then washed five times for 5 min each time with 50 mM Tris-HCl, pH 7.5, 200 mM NaCl, and 2% Triton X-100, and incubated in the same buffer containing 5% nonfat milk (Blotto 2) for 30 min at room temperature. The filters were then incubated for 1 h at room temperature in Blotto 2 containing a 1:10,000 dilution of peroxidase-conjugated goat anti-mouse or anti-rabbit secondary antibody (Amersham). They were washed five times and ECL was performed according to the manufacturer's instructions. Determination of PKC Isozyme and bcl-x mRNA. Total cellular RNA was isolated using TRIZOL reagent (Life Technologies), total RNA resolved (20-30 μg) on 1.2% agarose gel containing 1.1% formaldehyde, and transferred to Hybond-N nylon membranes (Amersham). The human PKC-α cDNA probe (courtesy I. B. Weinstein) and the bcl-xL cDNA probe (courtesy S. Korsmeyer) were ⁵²P-radiolabeled with [α-³²P] dCTP by random primer labeling using a commercially available kit (Promega, Madison, Wis.) according to the manufacturer's instructions. The blots were then hybridized with these cDNA probes in 50% formamide, 5×SSC, 5×Denhard's solution, 0.5% SHS, 1% dextran sulfate, and 0.1 mg/ml of salmon sperm DNA overnight at 42° C. The filters were washed at room temperature twice for 15 min in 2×SSC and 0.1% SDS, once for 20 min in 1×SSC and 0.1% SDS, and finally twice for 15 min in 0.1×SSC and 0.1% SDS at 65° C. The filters were exposed to Kodak x-ray film with intensifying screens for 12-48 h at −70° C. and developed. Blots were then stripped and hybridization with a control GAPDH probe performed as above.

Confocal Microscopy. T24 cells were seeded in glass-bottom microwells, and treated with complexes of Lipofectin (10 μg/ml) and 1 μM Inno-1411 (5′-fluorescein-labeled Inno-1407) at 37° C. for 5 h in 120 μl wells. Cellular internalization was examined using an ISM 410 laser scanning confocal microscope (Zeiss, Thornwood, N.Y.) equipped with a krypton/argon laser and attached to a Zeiss Axiovert 100 TV microscope. The 515-540 nm bandpass for fluorescein was used. Z-series were taken of a 1-2 μm optical section at 2 μm intervals. For measurements, a maximum projection of all sections was employed. Images were printed using NIH Image 1.67.

TABLE I

Oligonucleotide Sequences Used In This Study
Underline denotes hybridizing nucleotides

| | |
|---|---|
| Oligomer | Nt+HZ,51 |

| | | |
|---|---|---|
| Isis 3521 | 5′-GTTCTCGCTGGTGAGTTTCA-3′ | 20⁽¹⁾ |
| Inno-1405 | C UCG CUG GAA GG(dU) U(rA)G (rA)(rA)U CCU UCG AGU UUC(iT) | 32⁽²⁾ |
| Inno-1406 | C UCG CUG GAA GG(dU) U(rA)G (rA)(rA)U CCU UCG AGU UUC [r(ACCA)] | 36⁽³⁾ |
| Inno-1407 | C UCG CUG GAA GG(dU) U(rA)G (rA)(rA)U CCU UCG AGU UUC ACCA (iT) | 36⁽⁴⁾ |
| Inno-1411 | F C UCG CUG GAA GG(dU) U(rA)G (rA)(rA)U CCU UCG AGU UUC ACCA(iT) | 36⁽⁵⁾ |
| Inno-1412 | C UCG CUG GUG AGU UUC (iT) | 17⁽⁶⁾ |
| Inno-1413 | C UCG CUG GAA GGU UAG AAU CCU UCG AGU UUC (iT) | 32⁽⁷⁾ |
| Inno-1414 | C UCG CUG GAA GGU (rA)(rA)G(rA) U(dU) CCU UCG AGU UUC(iT) | 32⁽⁸⁾ |
| Bcl-xL1 | A GCU GCG _{GAA GG}(dU) U(rA)G (rA)(rA)U CCU UCC CGA CUC(iT) | 32⁽⁹⁾ |
| Bcl-xL2 | A GCU GCG GAA GGU (rA)(rA)G (rA)U(dU) CCU UCC CGA CUC(iT) | 32⁽¹⁰⁾ |

Notes
⁽¹⁾All-phosphorothioate
⁽²⁾2′-O-methyl modified, same site as ISIS-3521 A/7 + D/7
⁽³⁾2′-O-methyl modified with all-RNA ACCA
⁽⁴⁾2′-O-methyl modified with all-2′-O-Me ACCA
⁽⁵⁾1407 with 5′-fluorescein
⁽⁶⁾All-2′-O-methyl antisense (control)
⁽⁷⁾2′-O-methyl control for Inno-1405
⁽⁸⁾Similar to Inno-1405 but with loop sequence reversed
⁽⁹⁾2′-O-methyl modified, targeted to bcl-xL mRNA nucleotides 623-638
⁽¹⁰⁾Same as bcl-xL1 but with inverted loop sequence

### REFERENCES

1. Stein, C. A. & Cheng, Y. C. Antisense oligonucleotides as therapeutic agents—is the bullet really magic? Science 261, 1004-1002 (1993).

2. Wagner, R. Gene inhibition using antisense oligodeoxynucleotides. Nature 372, 333-335 (1994).

3. Stein, C. A. & Krieg, A. eds. Applied antisense oligonucleotide technology. (Wiley-Liss, New York; 1998).

4. Mulamba, G., Hu, A., Azad, R., Anderson, K. & Coen, I. Human cytomegalovirus mutant with sequence-dependent resistance to the phosphorothioate oligonucletode fomivirsen (Isis 2922). Antimicrob. Agents Chemother. 42, 971-973 (1998).

5. Walder, R. Y., and Walder, J. A. Role of RNase H in hybrid-arrested translation by antisense oligonucleotides. Proc. Natl. Acad. Sci. USA 85, 5011-5015 (1988).

6. Minshull, J., and Hunt, T. The use of single-stranded DNA and RNase to promote quantitative “hybrid arrest of translation” of mRNA-DNA hybrids in reticulocyte lysate cell-free translations. Nucl. Acids Res. 14, 6433-6451 (1986).

7. Hausen, P., and Stein, H. Ribonuclease H. An enzyme degrading the RNA moiety of DNA-RNA hybrids. Eur. J. Biochem. 14, 278-283 (1970).

8. Stein, C. A., Subasinghe, C., Shinozuka, K., and Cohen, J. Physicochemical properties of phosphorothioate oligodeoxyribonucleotides. Nucl. Acids Res. 16, 3209-3221 (1988).

9. Giles, R., and Tidd, D. Increased specificity for antisense oligodeonucleotide targeting of RNA cleavage by RNase H using chimeric methylphosphonodiester/phosphodiester structures. Nucl. Acids Res. 20, 763-770 (1992).

10. Giles, R., Spiller, D., and Tidd, D. Chimeric oligodeoxynucleotide analogues: Enhanced cell uptake of structures which direct ribonuclease H with high specificity. Anti-Cancer Drug Res. 8, 33-51 (1993).

11. Giles, R., Ruddell, C., Spiller, D., Green, J., and Tidd, D. Single base discrimination for ribonuclease H dependent antisense effects within intact human leukemia cells. Nucl. Acids Res. 23, 954-961 (1995).

12. Monia, B., Lesnik, E., Gonzalez, C., Lima, W., McGee, D., Guinosso, C., Kawasaki, A., Cook, P., and Freier, S. Evaluation of 2′-modified oligonucleotides containing 2′-deoxy gaps as antisense inhibitors of gene expression. J. Biol. Chem. 268, 14154-14522 (1993).

13. Benimetskaya, L., Takle, G., Vilenchik, M., Lebedeva, I., Miller, P., and Stein, C. A. Cationic Porphyrins: Novel delivery vehicles for antisense oligonucleotides. Nucl. Acids Res. 26, 5310-5317 (1998).

14. Forster, A., and Altman, S. External guide sequences for an RNA enzyme. Science 249, 783-786 (1990).

15. Altman, S. RNA enzyme-directed gene therapy. Proc. Natl. Acad. Sci. USA 90, 10898-10900 (1993).

16. Ma, M., Jacob-Samuel, B., Dignam, J., Pace, U., Goldberg, A., and George, S. Nuclease-resistant external guide sequence-induced cleavage of target RNA by human ribonuclease P. Antisense Nucl. Acid Drug Devel. 8, 415-426 (1998).

17. Heidenreich, O., Benseler, F., Fahrenholz, A., & Eckstein, F. High activity and stability of hammerhead ribozymes containing 2′-modified pyrimidine nucleosides and phosphorothioates, J. Biol. Chem. 269, 2131-2138 (1994).

18. Yuan, Y., Hwang, E., and Altman, S. Targeted cleavage of mRNA by human Rnase, P. Proc. Natl. Acad. Sci. USA 89, 8006-8010 (1992).

19. Dean, N., McKay, R., Condon, T., and Bennett, F. Inhibition of protein kinase C-α expression in human A549 cells by antisense oligonucleotides inhibits induction of intercellular adhesion molecule 1 (ICAM-1) mRNA by phorbol esters. J. Biol. Chem. 269, 16416-16424 (1994).

20. Dean, N., and McKay, R., Inhibition of protein kinase C-α expression in mice after systemic administration of phosphorothioate antisense oligodeoxynucleotides. Proc. Natl. Acad. Sci. USA 91, 11762-11766 (1994).

21. Loria, A., and Pan, T. Recognition of the T stem-loop of a pre-tRNA substrate by the ribozyme from Bacillus subtilis ribonuclease P. Biochemistry 36, 6317-6325 (1997).

22. Kahle, D., Wehmeyer, U., and Krupp, G. Substrate recognition by RNase P and by the catalytic M1 RNA: Identification of possible contact points in pre-tRNAs. EMBO J. 9, 1929-1937 (1990).

23. Werner, M., Rose, E., Al Emran, O., Goldberg, A., and George, S. Targeted cleavage of RNA molecules by human RNase P using minimized external guide sequences. Antisense Nucl. Acid Drug Devel. 9, 81-98 (1999).

24. Werner, M., Rosa, E., Nordstrom, J., Goldberg, A., and George, S. Short oligonucleotides as external guide sequences for site-specific cleavage of RNA molecules with human RNase P. RNA 4, 847-855 (1998).

25. Ortigao, J., Rosch, H., Selter, H., Frohlich, A., Lorenz, A., Montenarh, M., and Seliger, H. Antisense effect of oligodeoxynucleotides with inverted terminal internucleotidic linkages: A minimal modification protecting against nucleolytic degradation. Antisense Res. Devel. 2, 129-146 (1992).

26. Sinha, N., Davis, P., Usman, N., Perez, J., Hodge, R., Kremsky, J., and Casale, R. Labile exocyclic amine protection of nucleosides in DNA, RNA and oligonucleotide analog synthesis facilitating N-deacylation, minimizing depurination and chain degradation. Biochimie 75, 13-23 (1993).

What is claimed is:

1. A nuclease-resistant external guide sequence (EGS) oligonucleotide selected from the group consisting of Inno-1405, Inno-1407 and Bcl-xL1 whose sequences are set forth in Table 1.

2. A method of inducing specific intracellular mRNA cleavage through activation of RNase P comprising contacting a cell with a complex comprising an EGS oligonucleotide and a carrier.

3. The method of claim 2 wherein the EGS oligonucleotide is of a 0.1 μM/ml to 1 μM/ml concentration and the carrier is of a 1 μg/ml to 30 μg/ml concentration.

4. The method of claim 2 wherein the carrier is a cationic lipid.

5. The method of claim 2 wherein the cationic lipid is Lipofectin or LipofectACE.

6. The method of claim 2 wherein the EGS oligonucleotide is selected from the group consisting of Inno-1405, Inno-1407 and Bcl-xL1 whose sequences are set forth in Table 1.

7. The method of claim 2 wherein the cell is a human cell.

8. The method of claim 7 wherein the human cell is selected form the group consisting of a prostate cell, bladder cell, colon cell, breast cell, lung cell, endometrial cell, epithelial cell, ovarian cell, cervical cell, neural cell and blood cell.

9. The method of claim 2 wherein the cell is a cancer cell selected from a group consisting of melanoma cells, basal cell carcinoma cells, squamous cell carcinoma cells, neuroblastoma cells, glioblastoma multiforme cells, myeloid leukemic cells, breast carcinoma cells, colon carcinoma cells, endometrial carcinoma cells, lung carcinoma cells, ovarian carcinoma cells, prostate carcinoma cells, bladder cancer cells, cervical carcinoma cells, osteosarcoma cells and lymphoma cells.

10. The method of claim 2 wherein the RNase P does not cleave nontargeted mRNAs.

11. A method of downregulating PKC-α protein expression comprising contacting a cell with a complex comprising an EGS oligonucleotide and a carrier.

12. The method of claim 11 wherein the EGS oligonucleotide is selected from the group consisting of Inno-1405 and Inno-1407 whose sequences are set forth in Table 1.

13. The method of claim 11 wherein the EGS oligonucleotide is of a 0.1 μM/ml to 1 μM/ml concentration and the carrier is of a 1 μg/ml to 30 μg/ml concentration.

14. The method of claim 11 wherein the carrier is a cationic lipid.

15. The method of claim wherein the cationic lipid is Lipofectin or LipofectACE.

16. A method of downregulating bcl-xL protein expression comprising contacting a cell with a complex comprising an EGS oligonucleotide and a carrier.

17. The method of claim 16 wherein the EGS oligonucleotide is bcl-xL1 whose sequence is set forth in Table 1.

18. The method of claim 16 wherein the EGS oligonucleotide is of a 0.1 μM/ml to 1 μM/ml concentration and the carrier is of a 1 μg/ml to 30 μg/ml concentration.

19. The method of claim 18 wherein the carrier is a cationic lipid.

20. The method of claim 19 wherein the cationic lipid is Lipofectin or LipofectACE.

21. A method of downregulating PKC-α mRNA expression comprising contacting a cell with a complex comprising an EGS oligonucleotide and a carrier.

22. The method of claim 21 wherein the EGS oligonucleotide is selected from the group consisting of Inno-1405 and Inno-1407 whose sequences are set forth in Table 1.

23. The method of claim 21 wherein the EGS oligonucleotide is of a 0.1 μM/ml to 1 μM/ml concentration and the carrier is of a 1 μg/ml to 30 μg/ml concentration.

24. The method of claim 23 wherein the carrier is a cationic lipid.

25. The method of claim 24 wherein the cationic lipid is Lipofectin or LipofectACE.

26. A method of downregulating bcl-xL mRNA expression comprising contacting a cell with a complex comprising an EGS oligonucleotide and a carrier.

27. The method of claim 26 wherein the EGS oligonucleotide is bcl-xL1 whose sequence is set forth-in Table 1.

28. The method of claim 26 wherein the EGS oligonucleotide is of a 0.1 μM/ml to 1 μM/ml concentration and the carrier is of a 1 μg/ml to 30 μg/ml concentration.

29. The method of claim 28 wherein the carrier is a cationic lipid.

30. The method of claim 28 wherein the cationic lipid is Lipofectin or LipofectACE.
